(19) United States
(12) Patent Application Publication
(10) Pub. No.: US 2026/0358213 A1
(43) Pub. Date: Nov. 12, 2026

(54) INITIAL CONDITION EVALUATION FOR FIBRE SAMPLE

(71) Applicant: Conopco, Inc., d/b/a UNILEVER, Hoboken, NJ (US)

(72) Inventor: Kenneth Stuart LEE, Wageningen (NL)

(21) Appl. No.: 19/130,026

(22) PCT Filed: Dec. 8, 2023
(86) PCT No.: PCT/EP2023/084815

§ 371 Date: May 14, 2025

(30) Foreign Application Priority Data
Dec. 23, 2022 (EP) 22216313.1

(51) Int. Cl. G01N 3/32; G01N 33/483

(52) U.S. Cl. CPC G01N 3/32; G01N 33/4833

(57) ABSTRACT

An apparatus, method and associated computer program for determining the initial condition of a fibre sample. The method comprises: receiving fatigue testing data for the fibre sample for a plurality of loading cycles, in which the fatigue testing data comprises force or stress against displacement or strain data; determining, for at least a subset of the cycles, a loading energy based on data from the fatigue testing data; and determining an indication of initial condition in the fibre sample based on the loading energy of the at least a subset of the cycles.

### FIELD OF THE INVENTION

The disclosure relates to a method, and associated computer program code and apparatus, for evaluating an initial condition of a hair fibre sample during fatigue analysis. In particular, the method may relate to evaluating a human hair sample.

### BACKGROUND OF THE INVENTION

Mechanical testing, and in particular tensile testing and fatigue analysis, are used in a wide variety of industries to analyse the properties of products and materials to allow engineers to improve designs and chemical formulations.

In the fast moving consumer goods space, in developing hair care products, the effect of the product on the properties of hair fibres needs to be tested so that the effect of the product as a chemical treatment can be assessed and compared with that of existing formulations. Typically, such analysis may be performed by comparing mechanical properties of fibres with and without the product treatment being applied. A common mechanical parameter used for these comparisons is Young's modulus. This can be measured using a simple tensile test (measuring force vs strain, or stress vs strain). In some examples, the tensile test is repeated for approximately 50 fibres of each type (treated vs untreated). This type of test may be referred to as the ‘classical tensile test’. The overall experiment is relatively rapid and can be used to screen formulations to determine their effectiveness in affecting the physical properties of the fibre samples in a desired way.

However, fibre fatigue experiments are more relevant to the performance of some materials, including human hair, in real world conditions than stress/strain measurements. In some examples, fatigue measurements may be taken by repeatedly cycling straining and recovery for a sample. In hair care product testing, such analysis is more representative of the mechanical insult that hair fibres receive during grooming than classical tensile testing. Typically, this cyclic process may be repeated until the fibre breaks. This type of test may be referred to as ‘single fibre fatigue testing’. The average number of ‘cycles to break’ can be recorded for a number of fibres (for example 30 or 50 fibres, or more). It is also reported in the literature that differences between hair types may be much larger in fatigue measurements than in conventional measurements.

The present disclose addresses problems related to determining further useful physical attributes from such analysis methods.

### SUMMARY OF THE INVENTION

The disclosure relates to a method of determining an indication of the fatigue history of a fibre sample before a fatigue experiment, the method comprising:
receiving fatigue testing data for the hair fibre sample for a plurality of loading cycles;
determining, for at least a subset of the cycles, loading energy for each of the plurality of loading cycles based on the fatigue testing data; and
determining the indication of fatigue history of a hair fiber sample before a fatigue
experiment based on a function of the loading energy with respect to the number of cycles.

The method may be a computer-implemented method. The method may be performed at least in part using a digital processor.

The fibre sample is a hair sample. The fatigue testing data may be, or may comprise, force or stress against displacement or strain data.

Determining the indication of initial condition may comprise determining an indication of discontinuities in the function of the loading energy with respect to the number of cycles. Determining the indication discontinuities in the function of the loading energy may comprise determining one or more parameters associated with the function of the loading energy with respect to the number of cycles.

The parameters may comprise the location and/or number of peaks or local maxima. A graph of a value of the parameter vs fatigue cycle number may contain several maxima. These maxima are related to partial breakage events within the fibre. A fibre that is in better condition at the start of the fatigue experiment will be able to withstand more of these partial breakage events than a fibre that is in a poorer (more fatigued) state at the start of the experiment. These local maxima can therefore provide information on the past history of the fibre sample. The identification of peaks may comprise convolving the function of the loading energy with a continuous function. The identification of peaks may comprise differentiating the data resulting from the convolution. The identification of peaks may comprise locating the peaks from the differentiated data. The continuous function may be a Gaussian function.

The identification of parameters may comprise generating a synthetic function of loading energy with respect to the number of cycles. The identification of parameters may comprise comparing the synthetic function with the function of the loading energy. The identification of parameters may comprise varying the parameters of the synthetic function in order to match the function of the loading energy.

Determining the indication of the initial condition may comprise generating a structure function having scaled delta functions indicating the start of the decay process and/or the location of each of the discontinuities or peaks. Determining the indication of the initial condition may comprise determining a measure of the structure or information in the of the structure function. Determining the indication of the initial condition may comprise the number of scaled delta functions in the structure function. Determining the indication of the initial condition may comprise determining a total intensity in the structure function. Determining the indication of the initial condition comprises determining an entropy of the structure function. In these examples, intensity and entropy may be used in the sense understood in mathematics or information theory.

The loading energy of a loading cycle may be determined by integrating force/stress over displacement/strain for the loading portion of that loading cycle. The loading energy of a cycle may further comprise integrating force or stress over displacement or strain of the unloading part of that cycle to determine the energy lost within that cycle. Each discontinuity may be representative of a partial breakage event.

The fatigue testing for a plurality of loading cycles may be performed using an automated testing schedule. Each hair fibre sample may comprise a human or animal hair fibres.

The method may comprise performing the fatigue testing for the plurality of loading cycles.

According to a further aspect, there is provided a fatigue testing apparatus comprising a controller configured to, when in use, perform a method disclosed herein. According to a further aspect, there is provided a computer readable storage medium comprising computer program code configured to, when in use, cause a processor to perform a method disclosed herein. According to another aspect, the disclosure provides a computer program, distributable by electronic data transmission, comprising computer program code means adapted, when said program is loaded onto a computer, to make the computer execute the procedure of any of the methods described herein, or a computer program product, comprising a computer readable medium having thereon computer program code means adapted, when said program is loaded onto a computer, to make the computer execute the procedure of any one of methods described herein.

According to a further embodiment of the disclosure, there is provided a fatigue testing apparatus comprising a controller configured to, when in use, perform one or more of the methods described herein.

### BRIEF DESCRIPTION OF THE DRAWINGS

One or more embodiments will now be described by way of example only with reference to the accompanying drawings in which:

FIG. 1 shows a method of evaluating an initial condition of a fibre sample;

FIG. 2 shows a plot of force-strain hysteresis curve for a fibre loading cycle;

FIG. 3 shows a plot of measured loading energy as a function of cycle number;

FIG. 4 shows a plot of measured loading energy as a function of cycle number for a sample with significantly more partial breakage points than that in FIG. 3;

FIG. 5 shows results of a ‘cycles to break’ experiment comparing virgin hair samples and hair samples that have been heat treated;

FIGS. 6A to 6C shows results of applying the analysis method described previously with reference to FIGS. 1 to 4 comparing virgin hair samples and hair samples that have been heat treated;

FIG. 7 shows a plot of data from an example fatigue experiment comparing virgin hair samples and hair samples that have been heat treated;

FIG. 8 shows a plot of data from the example fatigue experiment corresponding to FIG. 7;

FIG. 9 shows a schematic block diagram of a system for performing a method of evaluating fatigue in a fibre sample; and

FIG. 10 shows a computer readable medium comprising computer program code.

### DETAILED DESCRIPTION OF THE INVENTION

Aspects of the present disclosure relate to evaluating the fatigue history of a hair fibre sample analysed in fatigue analysis, which enables the fatigue history to be taken into account when comparing results. For example, in fibre fatigue testing, such as the single fibre fatigue testing described previously, the number of cycles to break is influenced by two processes:
a) Degradation of the fibres during the fatigue experiment (related to current fibre resilience); and
b) Degradation of the fibres during their previous fatigue history (related to previous damage and any remediating effect of treatments).

In an ideal world, fatigue experiments would be carried out using pristine fibres with no existing fatigue damage. The number of cycles to break in such experiments would reflect only the degradation of the fibres during the fatigue experiment. However, for hair fibres, this is not possible because each fibre will have undergone some level of fatigue during washing, grooming etc. Variability in the treatments experienced by each fibre during the whole of its growth prior to harvesting results in a wide range of fatigue conditions prior to the fatigue experiment. This can considerably increase the scatter on the data from the experiment.

It may be possible to improve the amount of information that can be obtained from a fatigue experiment on a hair fibre by separating out the following two processes and providing information on each:
a) Degradation of the fibres during the fatigue experiment, and
b) Degradation of the fibres during their previous fatigue history.

It may be possible to assess the previous fatigue history of hair fibres by carrying out detailed analysis looking for breaks in the internal structure. Various microscopy and tomography techniques could be used. However, such approaches would have drawbacks, including:
1) Any technique requiring sectioning or staining of the fibre would prevent that fibre from being used in subsequent fatigue testing. While this approach could be used to characterise the state of fatigue of a sample of hair fibres, a separate sample would be needed for the fatigue experiment.
2) Microscopy and tomography experiments can be time consuming and expensive. Analysis of sets of 40 fibres would therefore not be practical for anything but a very limited study.

Some aspects of the present disclosure are directed to ways of identifying the initial physical properties of the fibre sample under test. This may comprise separating the effects of fatigue within the experiment from the previous fatigue state of the sample, using only the data generated from the fatigue experiment itself. More generally, the present disclosure relates to identifying the initial physical properties of the fibre sample under test. In some cases, the fibre samples may comprise human or animal hair fibres to allow for the efficient comparative testing of damage treatments, such as heat treatments, or chemical treatments, such as different shampoos or other consumer product formulations. Alternatively, the fibre samples may comprise natural hair fibres.

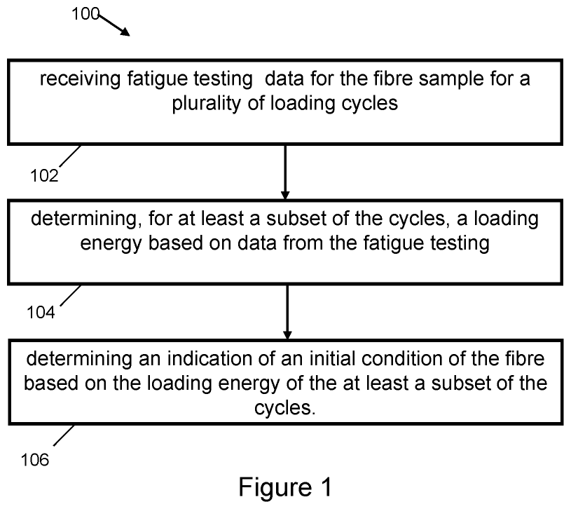

FIG. 1 outlines an example method 100 for determining the indication of the fatigue history of a hair fibre sample before a fatigue experiment. The method comprises receiving 102 fatigue testing data for the fibre sample for a plurality of loading cycles. The fatigue testing data may comprise force or stress against displacement or strain data. In some examples, the method can include performing the fatigue testing to obtain the fatigue testing data, and it will be appreciated that in practice the testing may be performed for a plurality of different samples in various conditions to allow a comparison between the performance of one or more products. For at least a subset of the cycles, a loading energy is determined 104 based on data from the fatigue testing. An indication of an initial condition of the cycle is then determined 106 based on the loading energy of the at least a subset of the cycles. In some examples, determining the fatigue history of a hair fibre sample comprises determining an indication of discontinuities in the function of the loading energy with respect to the number of cycles. It has been found that identification of peaks in the function can provide information regarding the initial state that the fibre was in before being subjected to fatigue testing. In summary, the quantified measurements regarding the fatigue history of a hair fibre sample may involve a number of different types of analysis of the function of the loading energy with respect to the number of cycles, which relates to generally determining a measure of the structure or information (in the sense of information theory) in the structure function. In specific terms, a quantification of the fatigue history of a hair fibre sample may be made by:
counting the number and/or determining the location of peaks, or local maxima;
finding the area under the structure function;
measuring entropy of the structure function; or
measuring intensity in the structure function.

By “determining the location of peaks” it may be meant that determining the indication of the fatigue history of a fibre sample includes taking account of where peaks occur in a function of the loading energy with respect to the number of loading cycles when quantifying the fatigue history of a hair fibre sample. In some embodiments, calculations of the fatigue history of a hair fibre may take account of after how many loading cycles a peak/partial breakage event in the loading energy has occurred. For example, quantifying the fatigue history of a hair sample may comprise determining a function comprising several delta functions multiplied by different constants indicating the location of the partial breakage events in the fatigue testing data.

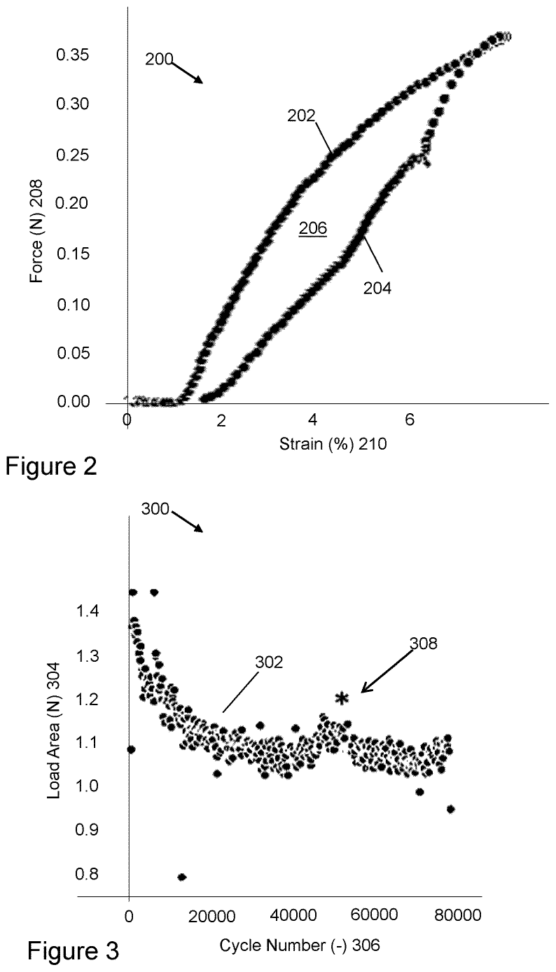

Various aspects of such methods are discussed in further detail below with reference to FIGS. 2 to 8.

As will be appreciated from the discussion below, various aspects of the method allow the evolution of a specific parameter that changes with the number of fatigue cycles applied to be studied. In some examples, the result for each set of samples may be correlated with the conventional fatigue measurement metric of the number of cycles to breakage.

FIG. 2 shows an example force-strain hysteresis curve of a loading cycle 200 for a single loading cycle. The X-axis shows the strain 210 experienced by the fibre sample and the Y-axis represents the force 208 exerted on the sample. Alternatively, the Y axis could show the stress and the X-axis the strain and yield a similar result. The force applied to the fibre sample induces the stress in the material under test. The data captured for the selected cycle includes a complete loading part 202 (upper curve) of the cycle 200 and complete unloading 204 part (lower curve) of the cycle 200 for the sample. The experiments were carried out using extension to a pre-determined stress. The loading part of the cycle ends when this stress is reached. Other implementations of the experiment could use a fixed force or a fixed strain.

The area under the force/strain loading part 202 of the cycle 200 provides a measure of the loading energy. The loading energy of a given cycle may be determined by integrating the force/stress over displacement/strain for the loading portion 202 of that cycle. The loading energy changes with the number of loading cycles that are applied to the fibre sample. The loading energy parameter can be used to determine the number of cycles required for the fibre sample to break. Monitoring the loading energy can therefore be used to provide a predictable assessment of the fatigue of the fibre sample. For example, while the analysis does not necessarily allow a prediction of breakage to be performed on a fibre-by-fibre basis, a comparison of the fatigue performance can be made between fibre samples based on the determined values. Fibre samples in which the magnitude of the rate of change of loading energy with respect to cycle number is greater indicate a higher rate of fatigue than fibre samples in which the rate of change is lower.

The fatigue testing data can be acquired using existing commercially available instruments using standard data acquisition parameters and techniques. The fatigue data may be obtained using a Dia-Stron CYC801. In a conventional experiment only the number of cycles to break is recorded. Whereas, in the disclosed method, a loading energy is determined based on data from the fatigue testing for at least a subset of the cycles. In the illustrated data, the force/strain curves are recorded during selected cycles (every 200^{th} cycle in this example) of the fatigue experiment.

The area 206 between the loading part 202 and the unloading part 204 indicates the amount of the energy lost during the loading cycle 200. The lost energy indicated by area 206 is equivalent to the area under the loading curve 202 (loading energy) minus the area under the unloading curve 204 (unloading energy).

Additional parameters derived from the loading energy may also be used to determine a function of the loading energy. Using the area under the loading part 202 of the cycle 200, or the area under the unloading part 204 of the cycle 200 or the area 206 within the hysteresis curve, the energy lost through the fibre sample during that cycle can be determined. The energy lost in the cycle can be used as a parameter to give a predictive indication of fibre breakage. More particularly, the changes to the shape of the loading part 202 of the cycle 200, the unloading part 202 of the cycle 200 and the area 206 within the hysteresis curve determined for each of the selected cycles can allow for an indicator of the likelihood of fibre breakage, or fatigue, to be determined.

FIG. 3 shows a time series profile 300 including selected data samples 302 for a physical specimen (or fibre sample). The profile 300 shows load area 304 against cycle number 306 for each selected sample 302. Each point in FIG. 3 represents the loading energy for a selected cycle, which may be determined for each point using the measurement described previously with reference to FIG. 2, for example. Alternatively, each point may correspond to a parameter derived from the loading energy, such as the energy lost during a cycle (loading energy minus unloading energy).

It has been found that fatigue is demonstrated by changes in the loading curve over a number of loading cycles. It will be appreciated that the term loading cycle takes its conventional meaning when used herein: a cycle in which a load is applied and removed. The evolution of the area under the loading curve can therefore be used as a predictive parameter for failure, and as an indicator of fatigue. The trends of the data in FIG. 3 can be used to provide an indication/prediction of the fibre breakage before the complete breakage of the fibre is experienced. For example, as shown in FIG. 3, initially, the loading energy (as indicated by the loading area 304) decreases as the number of cycles 306 increases. In particular the loading energy decreases as the log of the number of cycles 306.

As the number of cycles 306 is increased, the fibres are weakened and as such the amount of energy lost during each cycle (i.e., the load area 304 under the loading curve) also decreases. This can be considered as the fibre sample becoming easier to deform by the applied force.

The indication of the rate of fatigue may comprise determining a rate of change of the loading energy with respect to the number of cycles. In particular, it has been found that the initial slope of the loading energy with respect to the number of cycles may provide a useful indicator of fatigue. The indication of the rate of fatigue may be determined based on initial loading cycles. This may be determined by comparing the energy lost in relation to the initial state of the fibre samples, for example, when the load area has reduced by a threshold level, such as 10% or 20%. Alternatively, the initial loading cycles may include a fixed number of cycles, such as 5,000, 10,000, 20,000 or 50,000 cycles, for example. As a further alternative, the number of loading cycles to provide the initial loading cycles may be based on determining when the rate of change of the initial slope of the loading energy with respect to the number of cycles reaches a threshold value.

The rate of change of the loading energy may be based on smoothed values of the loading energy 304 for the plurality of cycles 306. The application of a conventional smoothing function is useful to remove sample-to-sample variation and improve the conformity of a rate of change calculated based on the smoothed function with the overall trend. That is, a first derivative of the smoothed data provides an effective rate of decrease of the load area vs cycle number.

As shown in FIG. 3, the data also contains an unexpected bump 308 in the curve. These bumps are understood to correspond to partial breakage events of the fibre.

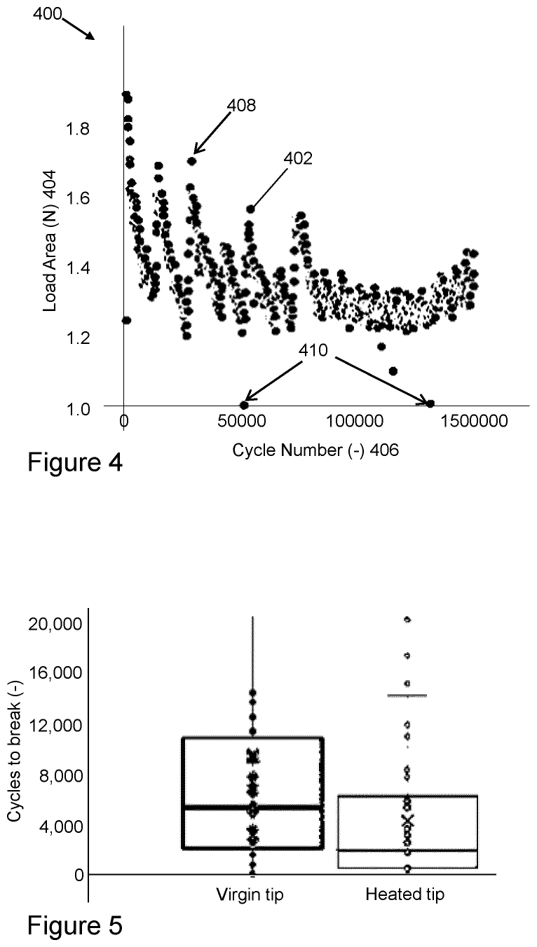

FIG. 4 illustrates another example time series profile 400 of load area 404 against cycle number 406 of a similar type to that described previously with reference to FIG. 3. In FIG. 4, the data shows a lot of unexpected bumps 408 compared to the sample testing in FIG. 3. As discussed previously, these bumps, or outliers, are understood to correspond to partial breakage events.

A number of erroneous data points 410 are also included in FIG. 4 which are of no physical significance. These datapoints may be screened out of analysis by thresholding, for example.

Differences in the number of detected partial breakage events in the profile 400 of a fibre sample is understood to indicate that the fibre sample was in a different initial state, or initial condition, compared to the other fibre samples. By initial state, it is meant the state of the fibre before fatigue testing. The initial condition therefore includes the total past history of the fibre, including the effects of all grooming events and treatments prior to the measurement. Aspects of the initial condition may include fibre strength, integrity, physical damage (such as splits in the fibre) and moisture concentration, for example. The initial state can be representative of how much damage the fibre sample has received prior to the fatigue testing. An initially more intact fibre may be able to survive more partial breakage events before completely breaking. Therefore, a greater number of unexpected bumps 408 may represent a fibre with a less damaged initial state in which the fibre was initially more intact than a fibre that only gives a small number of bumps.

That is, a fibre showing more of the bumps 408 during data acquisition can be considered as being in a better initial state than one with fewer bumps. A fibre in a better initial state can therefore survive more small/partial breakage events during the experiment prior to the whole fibre breaking or weakening to a point where the loading energy reaches the threshold level.

The observation of the number of partial breakage events for a fibre provides an indicator of the initial state of the fibre that is not available from conventional testing. That is, in a conventional test the initial state of the fibre samples cannot be factored in and can lead to a range of measured values of fatigue for the fibre samples.

Therefore, by counting the number of partial breakage events (either to failure or up to a predetermined number of cycles, such as 100000), a measure of existing fibre damage defining the initial state of the fibre sample may be provided. In this way, the number of partial breakage events detected in the data of FIG. 3 or FIG. 4 may be considered to provide the indication of the initial state of the fibre sample undergoing testing.

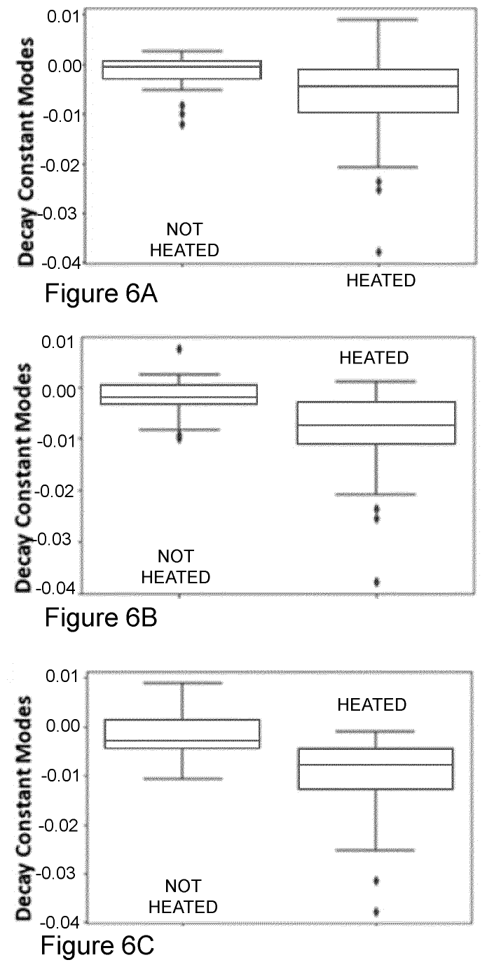

FIGS. 5 and 6 compare data obtained using conventional fatigue measuring experiments and a method in which cycling fatigue data is used to provide an indication of fatigue. The experiments also demonstrate qualitatively that the fatigue history of hair fibres have an effect on fatigue analysis findings.

The samples used in the experiment were taken from the following hair types:
a) One tress of virgin blended medium brown Caucasian hair, and
b) One tress of heat treated (30 passes at 450 °F (approx. 232.2 °C) using hair styling irons) blended medium brown Caucasian hair.

Virgin hair was supplied by International Hair Importers. For both hair types, samples were taken from tip ends of the fibre. There are therefore 2 sample types.

Fatigue testing was conducted at 80% relative humidity (80% RH) at room temperature using a cyclic tester (Dia-Stron CYC801) in “constant stress mode”. The fatiguing rate was set 60 mm/s and the number of cycles to break was measured at 80% relative humidity using a target stress of 105 MPa. Note that both force and stress were recorded in these experiments (stress=force/cross sectional area). However, because fatigue processes will change the effective cross section of the fibre (the active cross section of the fibre will decrease as a function of crack propagation), the analysis is carried out in terms of the force/strain curve. This also removes the necessity to correct the data using the gross fibre cross-section dimensions. This is legitimate in this application because we are studying the evolution of parameters between cycles for the same fibre. To calculate the stress, the dimensions of fibres were measured at 60% RH (at five points along the fibre).

Approximately 40 fibres were used in each experiment. Instrumental problems resulted in there being fewer data sets for some of the cells of the experiment. Also, because of a limitation on the quantity of data that can be saved during one experiment, some fibres (those requiring a very large numbers of cycles to break) had to be re-run, resulting in more than one data file per fibre.

FIG. 5 shows the results of a ‘cycles to break’ experiment comparing virgin hair samples vs hair samples that have been heat treated. In both cases the hair was taken from the tip end of the switch.

FIG. 6 shows results of applying the analysis method described previously with reference to FIGS. 1 to 4 to A) the full data set (all of the cycles to break data), B) the first 100 data points, and C) the first 50 data points.

As is appreciable from a comparison of FIGS. 5 and 6, there is a similar qualitative change in the fatigue performance of the hair in both the heated and not heated conditions using the classical and new analysis methods.

The results in FIGS. 6A to 6C also show that good discrimination between the samples can be achieved for all three data sets. Hence, satisfactory results can be obtained using only the first 50 data points, for example. This represents a 7-fold reduction in the quantity of data required to obtain the experimental results shown in FIG. 6C compared to the conventional fatigue measurement described with reference to FIG. 5.

The data processing steps for preparing FIG. 6 are discussed below. The first derivative has been calculated for each time point, as previously described. The resulting data have been formed into a histogram of counts vs value of first derivative. In this example, 100 bins have been used for the values of the first derivative, although this could be varied. The bin with the greatest number of counts has been determined and the mid-point of that bin has been assigned to the decay constant. This process has been carried out for each fibre in the set to give a distribution of modes of the decay constant for each fibre set. The histogram is obtained using matplotlib.pyplot.hist, a Python package (see https://matplotlib.org/stable/api/_as_gen/matplotlib.pyplot.hist.html). The maximum of the histogram is found using the numpy. argmax Python package (see https://numpy.org/doc/stable/reference/generated/numpy.argmax.html).

An example approach according to the present methods uses the following equations to characterise the load area vs cycle number data,

f(x)=I(x)*(1−c.log(x+1)) [Eq. 1]

f(x)=I(x)*d [Eq. 2]

Where x=fatigue cycle number, d=1−c.log (x+1) (where c is the decay constant), * is the convolution operator and I(x) is an underlying structure function of the data, containing information on the start of the decay process. For the simplest case, I(x) should be a delta function at the first data point multiplied by a constant. In more typical cases, I(x) will include several delta functions multiplied by different constants, indicating the start of the decay process and each of the partial breakage events.

The structure function I(x) can be found by differentiation, following smoothing by convolution with a function g(x) that has a finite derivative for all values of x, e.g., a Gaussian function.

f(x)*g(x)=g(x)*I(x)*(1−c.log(x+1)) [Eq. 3]

Using the properties of the convolution operator,

d(f(x)*g(x))/dx=(dg(x)/dx)*I(x)*(1−c.log(x+1)) [Eq. 4]

This function exhibits peaks which approximate to I(x) multiplied by a constant w (i.e., w.I(x)).

Having determined w.I(x) from the preceding steps, to determine the decay constant c Eq. 3 can be re-written as,

f(x)*g(x)=(g(x)*I(x)*1)−c. (g(x)*I(x)*log(x+1)) [Eq. 5]

f(x)*g(x)=(∫g(x)*I(x)dx)−c. (g(x)*I(x)*log(x+1)) [Eq. 6]

w.I(x) is convoluted with g(x) and integrated to give J(x),

J(x)=w.∫g(x)*I(x)dx [Eq. 7]

Similarly, w.I(x)*g(x)*log(x+1) is determined,

w.I(x)*g(x)*log(x+1)=L(x) [Eq 8]

f(x) is fitted to J(x) and L(x),

f(x)*g(x)=p.J(x)−q.L(x) [Eq 9]

and c is found according to c=q/p.

The structure function I(x) provides information on the initial state of the fibre. Characterisation of I(x) may be carried out in various ways such as by counting the number of peaks, or by calculating the total area under I(x). A preferred characterisation is the entropy of w.I(x). In this way, the process provides a quantitative value related to the initial condition of the fibre.

An example process for analysing each fibre may include some or all of the following stages:
1. Data Acquisition using a commercial fatigue tester (e.g., those available from Dia-Stron).
2. Data Transfer to a laptop computer.
3. Data Processing, e.g., using Python in Jupyter Notebook.
3.1 Data visualisation and clean up.
3.2 Prepare the data for each cycle:
   Find threshold in Force/Strain data.
   Find area under loading part of Force/Strain curve=‘Loading Area’.
3.3 Construct response: Loading Area vs Number of Cycles.
3.4 Convolute the response data (f(x)) with a Gaussian function (or other smooth function), g(x), and take a local first derivative at each data point to give a decay constant. This may be achieved using a Savitzky-Golay filter (e.g., a signal. savgol filter from scipy signal).
3.5 Identify the location and maxima in the derivative found in 3.4, then construct the functions w.I(x), w.I(x)*g(x) and J(x).
3.6 Calculate the entropy of w.I(x). This may be achieved using a scipy.stats.entropy function from the scipy.stats library.
3.7 Calculate w.I(x)*g(x)*log(x+1)=J(x).
3.8 Fit f(x)*g(x) to p.J(x)−q.L(x) and determine c from c=q/p. This can be achieved using scipy.optimize.minimize.

For each set of data (where one set of data has several fibres, for example 40, per fibre-treatment*2 fibre-treatments), compare data between pairs of fibre-treatments using a non-parametric analysis such as the Mann-Whitney U test (scipy.stats.mannwhitneyu).

The example process may expedite pre-processing of thousands of data points for each of several thousands of testing cycles and/or carrying out convolution, differentiation, integration, and fit optimisation for hundreds of data sets. Advantageously, the calculations can be performed over a timescale that complements a corresponding cycles-to-break experiment, rather than over a timescale that prevents practical implementation. Furthermore, the calculation error rate will be improved over (i.e., lower than) alternative, manual-led approaches where the probability of generating errors would likely be high.

In other words, carrying out the calculations using a computer, the calculations can be conducted fast enough to be useful for some practical applications. It also allows the possibility of processing in real time i.e., processing the data from each fatigue cycle immediately after acquisition. This would allow quality control to be implemented during the experiment. This could also lead to intelligent stopping of the experiment once the data indicated that some pre-defined level of quality and consistency has been achieved. This approach could lead to further time saving.

Further data processing steps can be taken for extracting more information from the data, specifically information relating to the initial state of the fibres.

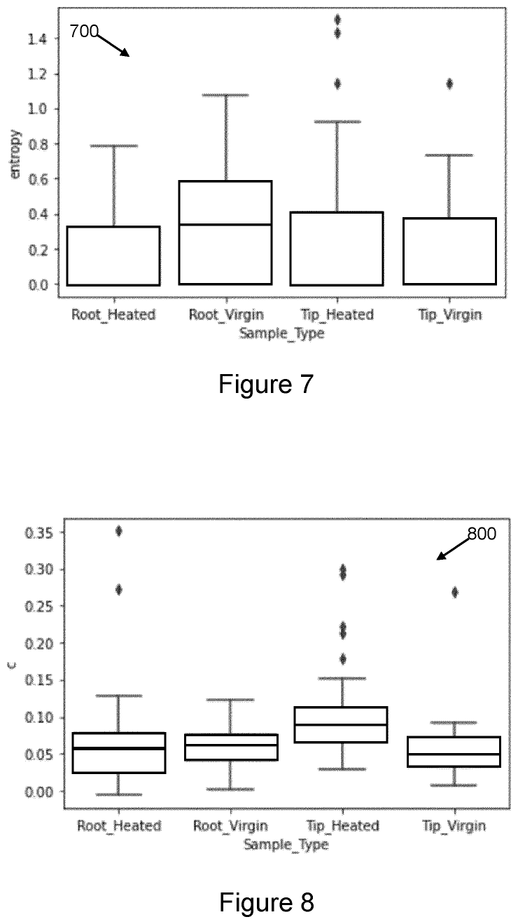

FIGS. 7 and 8 show plots of data 700, 800 from an example fatigue experiment comparing virgin hair samples and hair samples that have been heat treated. In this example fatigue experiment, a hair sample was taken from a) one tress of virgin blended medium brown Caucasian hair, and b) one tress of heat treated (30 passes at 450 °F (approx. 232.2 °C) using hair styling irons) blended medium brown Caucasian hair at the root end and at the tip end of each tress. Virgin hair was supplied by International Hair Importers. Four hair samples were therefore subject to analysis: root hair without heat treatment (‘Root_Virgin’); root hair following heat treatment (‘Root_Heated’); tip hair without heat treatment (‘Tip_Virgin’); and tip hair following heat treatment (‘Tip_Heated’).

By conducting the analysis described previously, and at a confidence interval of 95%, the ‘Root_Virgin’ hair sample was found to have a larger entropy (reflecting a more pristine sample state before the start of the fatigue experiment) than the ‘Root_Heated’ hair sample. These two hair samples did not differ in their exhibited values of decay constant c, however, reflecting similar rates of degradation during the fatigue experiment. The ‘Tip_Heated’ hair sample was found to have the largest (again, at 95% confidence) decay constant c of all four hair samples, reflecting greatest degradation during the fatigue experiment compared with the other samples ‘Root_Virgin’, ‘Root_Heated’ and ‘Tip_Virgin’.

The analysis described previously therefore allows the following two processes to be advantageously discriminated or separated: degradation of hair fibres during a current fatigue experiment, and degradation of the same hair fibres induced by earlier fatigue (i.e., during their previous fatigue history).

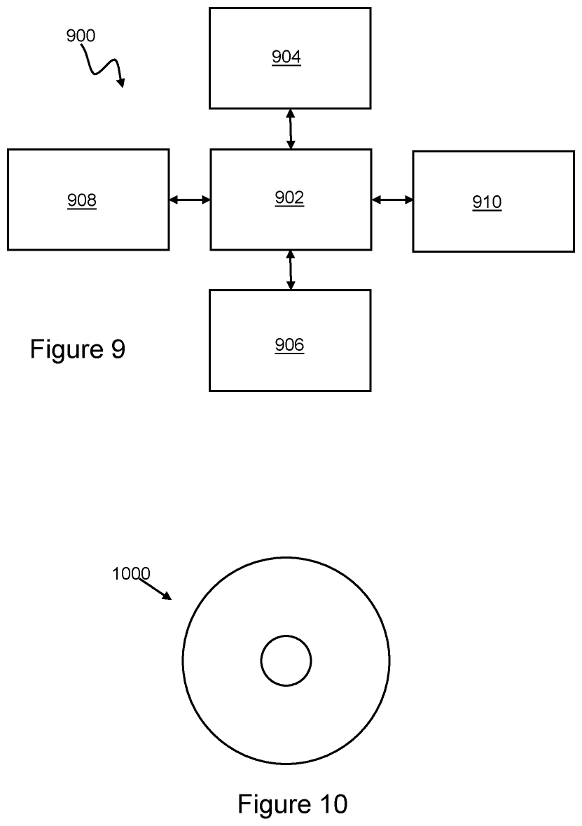

FIG. 9 illustrates a schematic block diagram of a system 900 which may be used to implement the methods described previously. The system 900 comprises one or more processors 902 in communication with memory 904. The memory 904 is an example of a computer readable storage medium. The one or more processors 902 are also in communication with one or more input devices 906 and one or more output devices 908. The various components of the system 900 may be implemented using generic means for computing known in the art. For example, the input devices 906 may comprise a keyboard or mouse and the output devices 908 may comprise a monitor or display, and an audio output device such as a speaker. In addition, the system 900 comprises a fatigue testing apparatus, which may be conventional, such that the fatigue testing apparatus 910 is at least partially under the control of the one or more processors 902.

FIG. 10 discloses a non-transitory computer readable storage medium 1000. The non-transitory computer readable storage medium comprises computer program code configured to cause a processor to perform a method disclosed herein.

In this specification, example embodiments have been presented in terms of a selected set of details. However, the skilled person would understand that many other example embodiments may be practiced which include a different selected set of these details. It is intended that the following claims cover all possible example embodiments.

1. A method of determining an indication of a fatigue history of a hair fibre sample before a fatigue experiment, the method comprising:
receiving fatigue testing data for the hair fibre sample for a plurality of loading cycles;
determining, for at least a subset of the cycles, loading energy for each of the plurality of loading cycles based on the fatigue testing data; and
determining the indication of fatigue history of the hair fiber sample before the fatigue experiment based on a function of the loading energy with respect to the number of cycles.

2. The method of claim 1, wherein determining the indication of the fatigue history comprises determining an indication of discontinuities in the function of the loading energy with respect to the number of cycles.

3. The method of claim 2, wherein determining the indication of discontinuities in the function of the loading energy comprises determining one or more parameters associated with the function of the loading energy with respect to the number of cycles.

4. The method of claim 3, wherein the parameters comprise a location and/or number of peaks.

5. The method of claim 4, wherein the identification of peaks comprises:
convolving the function of the loading energy with a continuous function;
differentiating data resulting from the convolution; and
locating the peaks from the differentiated data.

6. The method of claim 3, wherein the identification of parameters comprises:
generating a synthetic function of loading energy with respect to the number of cycles;
comparing the synthetic function with the function of the loading energy; and
varying parameters of the synthetic function in order to match the function of the loading energy.

7. The method of claim 2, wherein determining the indication of the fatigue history of the hair fibre sample before the fatigue experiment comprises generating a structure function having scaled delta functions indicating the location of each of the discontinuities.

8. The method of claim 7, wherein determining the indication of the fatigue history of the hair fibre sample before the fatigue experiment comprises determining a measure of the structure or information in the structure function.

9. The method of claim 8, wherein determining the indication of the fatigue history of the hair fibre sample before the fatigue experiment comprises the number of scaled delta functions in the structure function.

10. The method of claim 8, wherein determining the indication of the fatigue history of the hair fibre sample before the fatigue experiment comprises determining a total intensity in the structure function.

11. The method of claim 8, wherein determining the indication of the fatigue history of the hair fibre sample before the fatigue experiment comprises determining an entropy of the structure function.

12. The method of claim 1, wherein the loading energy of a loading cycle is determined by integrating force/stress over displacement/strain for the loading portion of that loading cycle.

13. The method of claim 12, wherein the loading energy of a cycle further comprises integrating force or stress over displacement or strain of the unloading part of that cycle to determine an energy lost within that cycle.

14. The method of claim 1, wherein the fibre sample comprises a human or animal hair fibre.

15. A non-transitory computer readable storage medium comprising computer program code configured to, when in use, cause a processor to perform the method of claim 1.
